(19) United States
(12) Patent Application Publication
(10) Pub. No.: US 2024/0114861 A1
(43) Pub. Date: Apr. 11, 2024

(54) SEMI-DOMINANT MOLECULAR MARKER RELATED TO MAIZE DWARF AND APPLICATION THEREOF

(71) Applicant: Anhui Agricultural University, Hefei (CN)

(72) Inventors: Yang Zhao, Hefei (CN); Qing Ma, Hefei (CN); Xiaoduo Lu, Hefei (CN); Yixiao Wang, Hefei (CN); Hongying Wu, Hefei (CN)

(21) Appl. No.: 18/233,341

(22) Filed: Aug. 14, 2023

Related U.S. Application Data
(63) Continuation of application No. PCT/CN2023/099574, filed on Jun. 12, 2023.

(30) Foreign Application Priority Data
Oct. 10, 2022 (CN) 202211232107.X

(51) Int. Cl. A01H 1/04; C12Q 1/6895

(52) U.S. Cl. CPC A01H 1/045; C12Q 1/6895; C12Q 2600/13; C12Q 2600/156

(57) ABSTRACT

The present application discloses a semi-dominant molecular marker related to maize dwarf and an application thereof, and relates to the technical field of maize molecular breeding. The molecular marker related to the maize dwarf is caused by a non-synonymous mutation from G to A in a maize gene Zm00001d013465, and the molecular marker is a nucleotide sequence composed of the non-synonymous mutation site and its upstream and downstream bases. The present application identifies a dwarf mutant, coded as E5779, from an EMS mutation population with maize B73 as the genetic background. By genome resequencing and Mutmap mapping, it is found that the mutant is caused by the non-synonymous mutation from G to A in the gene Zm00001d013465. The molecular marker is developed based on this, and the analysis and utilization of E5779 are helpful to improve the maize plant height and density-tolerant breeding, it has an important breeding application potential.

### CROSS-REFERENCE TO RELATED APPLICATIONS

The present application is a continuation application of PCT application No. PCT/CN2023/099574 filed on Jun. 12, 2023, which claims the benefit of Chinese Patent Application No. 202211232107.X filed on Oct. 10, 2022. The contents of all of the aforementioned applications are incorporated by reference herein in their entirety.

### REFERENCE TO SEQUENCE LISTING

The Sequence Listing XML file submitted via the USPTO Patent Center, with a file name of “Sequence_listing_RONDA-23012-USPT.xml”, a creation date of Aug. 9, 2023, and a size of 9.0 KB, is part of the specification and is incorporated in its entirety by reference herein.

### TECHNICAL FIELD

The present application relates to the technical field of maize molecular breeding, in particular to a semi-dominant molecular marker related to maize dwarf and an application thereof.

### BACKGROUND

Maize is one of three important food crops in China, and occupies an extremely important position in industrial and agricultural production as well as national food security strategies. The yield of the maize is determined by the yield per plant and the planting density per unit area, and increasing the planting density is an effective way to improve the maize yield per unit area (Duvick, Maydica, 2005, 50(3):193-202). Due to the increase in planting density, the competition for water, fertilizer, light and other resources among plants is intensified, and the risk of lodging is increased, thus higher requirements are put forward for the plant architecture of density-tolerant maize, in which the maize plant height is one of the important traits concerned in the density-tolerant plant architecture. Creating dwarf or semi-dwarf mutants, identifying relevant dwarf genes, and applying it to breeding are important ways to conduct the breeding of density-tolerant varieties.

By cloning the “green revolution gene” semi-dwarf1 (sd-1) gene and reduced height 1 (rht1) of rice and wheat and applying it to breeding, the yield of crops is greatly improved (Sasaki et. al., Nature, 2002, 416(6882):701-702; Peng et. al., Nature, 1999, 400(6741):256-261). At present, some dwarf mutants discovered are mainly related to various hormones, in which it is mainly related to gibberellin synthesis and signal transduction, such as rice OsKS1, SLR1, GID2 genes (Gomi et. al., Plant J, 2010, 37(4): 626-634; Margis-Pinheiro et. al., Plant Cell Rep, 2005, 23(12): 819-833; Ikeda et. al., Plant Cell, 2001, 13(5): 999-1010), and maize Dwarf3, An1 genes (Winkler et. al., Plant Cell, 1995, 7(8), 1307-1317; Bensen et. al., Plant Cell, 1995, 7(1), 75-84). However, although some dwarf mutants are significantly reduced in plant height, it has many adverse traits, such as significant yield loss, so it is difficult to directly apply for breeding. Therefore, identifying excellent dwarf mutants with potential of breeding application is the key to genetic improvement of maize plant height and density-tolerant breeding.

### SUMMARY

A purpose of the present application is to provide a semi-dominant molecular marker related to maize dwarf and an application thereof, and to provide a new breeding pathway with more potential for genetic improvement of maize plant height and density-tolerant breeding.

The present application is achieved by the following technical schemes.

The present application provides a semi-dominant molecular marker related to maize dwarf, the molecular marker is caused by a non-synonymous mutation from G to A in a maize gene Zm00001d013465, the molecular marker is a nucleotide sequence composed of the non-synonymous mutation site and its upstream and downstream bases. Zm00001d013465 encodes a maize Dwarf9 gene, and has a nucleotide sequence as shown in SEQ ID NO.1. The molecular marker related to the maize dwarf has a nucleotide sequence as shown in SEQ ID NO.2, and the non-synonymous mutation site is located in the 130th site of SEQ ID NO.1 or SEQ ID NO.2, which is mutated from G of SEQ ID NO.1 into A of SEQ ID NO.2.

The present application further provides a specific primer for detecting the above molecular marker related to the maize dwarf, and its sequence is:

E5779-F:
(SEQ ID NO: 4)
5′-GAGCTCGGACAGCATGCTCT-3′;
E5779-R:
(SEQ ID NO: 5)
5′-TCCGTTTCCTTCCCAACTCCCAAT-3′.

The present application further provides a method for identifying a molecular marker related to maize dwarf using the above primer, and it includes the following steps: a genomic DNA of maize plant to be identified is used as a template, PCR amplification is performed by using the primer to obtain an amplification product, the amplification product is sequenced, and the base type of the non-synonymous mutation site of the molecular marker is determined.

The present application further provides an application of the above molecular marker related to the maize dwarf in breeding a new variety of dwarf and density-tolerant maize. The molecular marker has semi-dominance in a maize plant, the molecular marker is introduced into a plant of a variety to be improved or an inbred line plant by a field breeding or molecular breeding technology and the like, a heterozygous dwarf plant with a genotype AG or a homozygous dwarf plant with a genotype AA is obtained, and the plant height satisfies: homozygous dwarf plant<heterozygous dwarf plant<maize plant to be improved.

As a further optimization scheme of the present application, a method for breeding a new variety of dwarf and density-tolerant maize using the field breeding technology includes the following steps: a parent of the variety to be improved is hybridized with a variety containing the molecular marker related to the maize dwarf, to obtain an F₁-generation heterozygous dwarf plant, the F₁-generation heterozygous dwarf plant is self-crossed or back-crossed, and the homozygous dwarf plant or the heterozygous dwarf plant is acquired by trait separating and screening. Further preferably, the variety containing the molecular marker related to the maize dwarf is the maize E5779 dwarf mutant.

As a further optimization scheme of the present application, a method for breeding a new variety of dwarf and density-tolerant maize using the molecular breeding technology includes the following steps: a gene editing technology is used to mutate a base of the non-synonymous mutation site of the molecular marker in the genome of the variety to be improved from G into A, to obtain a dwarf variety.

The principle of the present application is that: the present application identifies a dwarf mutant, coded as E5779, from an EMS mutation population with maize B73 as the genetic background. Compared with the maize B73 inbred line, the trait of the E5779 mutant is caused by 1 base change, and it has greater prospect for marker development, germplasm identification, and breeding application.

Compared to existing technologies, the present application has the following advantages.

The present application identifies the dwarf mutant E5779 from the EMS mutation population with maize B73 as the genetic background. By genome resequencing and Mutmap mapping, it is found that the mutant is caused by the non-synonymous mutation from G to A in the gene Zm00001d013465, the non-synonymous mutation has the semi-dominant characteristics, and may significantly reduce the height of F₁-generation plants. The molecular marker is developed based on this, and the analysis and utilization of E5779 are helpful to improve the maize plant height and density-tolerant breeding, which has an important breeding application potential. The molecular marker developed on the basis of E5779 has the semi-dominant characteristics for the maize plant height, and thus the new maize variety with lodging resistance or density tolerance is bred, which has significant application prospect in production.

### BRIEF DESCRIPTION OF THE DRAWINGS

FIG. 1 shows phenotypic analysis of an E5779 mutant, and in the figure, A is plant phenotypes of wild-type B73 inbred line, B73×E5779 hybrid combination and E5779 mutant; B is statistics of plant height, ear height and tassel length of B73, E5779, and B73×E5779; C is statistics of stem diameter of the third internode above ground of B73, E5779, and B73×E5779; and D is statistics of tassel branch numbers of B73, E5779, and B73×E5779.

FIG. 2 shows gene mapping and position analysis of the E5779 mutant, and in the figure, A is identification to an SNP mutation site of the E5779 gene using a Mutmap mapping method; and B is structural analysis to the E5779 mutant gene.

FIG. 3 shows development of a molecular marker for the E5779 mutant, and in the figure, A is wild-type and E5779 mutant PCR amplification electrophoresis analysis; M. StarMarker D2000 Marker; 1-2, B73-1, and B73-2 amplified electrophoresis bands; 3-4, E5779-1, and E5779-2 amplified electrophoresis bands; and B is sequencing analysis of mutation sites in wild-type B73 and E5779 mutants.

FIG. 4 shows phenotypic analysis of hybrid combinations prepared by the E5779 mutant, and in the figure, A is plant phenotype analysis of CM37×B73 and CM37×E5779 hybrid combinations; B is statistical analysis of plant height and ear height of the CM37×B73 and CM37×E5779 hybrid combinations; and C is statistical analysis of stem diameter of the third internode above ground of the CM37×B73 and CM37×E5779 hybrid combinations.

FIG. 5 shows ear phenotype of a breeding application of the E5779 mutant.

### DETAILED DESCRIPTION OF THE EMBODIMENTS

Unless otherwise specified, the methods used in this embodiment are conventional methods known to those skilled in the art, and the reagents and other materials used are commercially available products.

B73 and CM37 inbred lines of maize used in this experiment are provided by the National Engineering Laboratory of Crop Stress Resistance Breeding, School of Life Sciences, Anhui Agricultural University.

The E5779 maize dwarf mutant used in this experiment comes from the EMS mutant library (http://elabcaas.cn/memd/public/index.html#/pages/search/geneid), and is a dwarf mutant identified from an EMS mutation population with maize B73 as a genetic background. The plant height and ear height of the mutant plant are significantly reduced, and the stem is thickened. By genome resequencing and Mutmap mapping, it is found that the mutant is caused by the non-synonymous mutation from G to A in the gene Zm00001d013465, the E5779 mutant has the semi-dominant characteristics, and may significantly reduce the height of F₁-generation plants. Therefore, E5779 has the important breeding application potential for improving the plant height and density-tolerant breeding.

The content of the present application is described in detail below in combination with specific implementation modes.

1. Identification and Phenotypic Analysis of Dwarf Mutant E5779

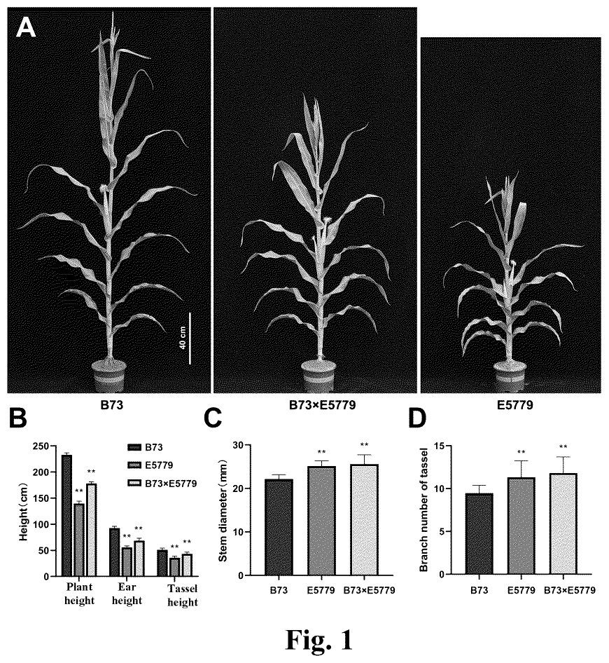

After being self-crossed, the dwarf mutant E5779 is shown in the same experimental field with the maize B73 inbred line, and after two generations of self-crossing observation, it is found that the phenotype of the E5779 mutant is consistent and genetically stable. As shown in A of FIG. 1, compared to the wild-type B73 inbred line, the plant height, ear height and tassel length of the E5779 mutant are all significantly reduced, it is found that the E5779 stem is thickened, which indicates that it has the potential to increase the plant lodging resistance.

2. Genetic Analysis of Dwarf Mutant E5779

The E5779 mutant is hybridized with the B73 inbred line before being self-crossed, and an F2 population obtained from self-crossing is shown in an experimental field with uniform field conditions. Around 15 days after the flowering period, the separation of traits in the F2 population is investigated by using the plant height as the main trait. It is found from results that in the F2 population, there are three types of plant height phenotypes: high, medium, and low, which represent wild-type B73, B73×E5779 heterozygous genotype and E5779 mutation genetic background respectively (the phenotypes are shown in A of FIG. 1, the plant height and ear height of the F₁-generation hybridization of B73×E5779 are between the B73 and E5779 mutants, which indicates that the E5779 mutant has the semi-dominant characteristics and great breeding application potential), the ratio is 41:92:43, and the separation ratio conforms to 1:2:1 (χ²=0.41<2χ_{0.05}²=5.99), which indicates that the E5779 is controlled by a single gene.

The plant height, ear height, tassel length, stem diameter of the third internode above ground, and tassel branching number of plants of wild-type B73, B73×E5779 heterozygous genotype and E5779 mutation genetic background are statistically analyzed, and results are shown in B-D of FIG. 1. The plant height, ear height, and tassel length of B73, B73×E5779, and E5779 are reduced sequentially, and the third internode stem diameter and tassel branching number of B73×E5779 and E5779 are significantly increased compared to B73.

3. Gene Mapping of Dwarf Mutant E5779

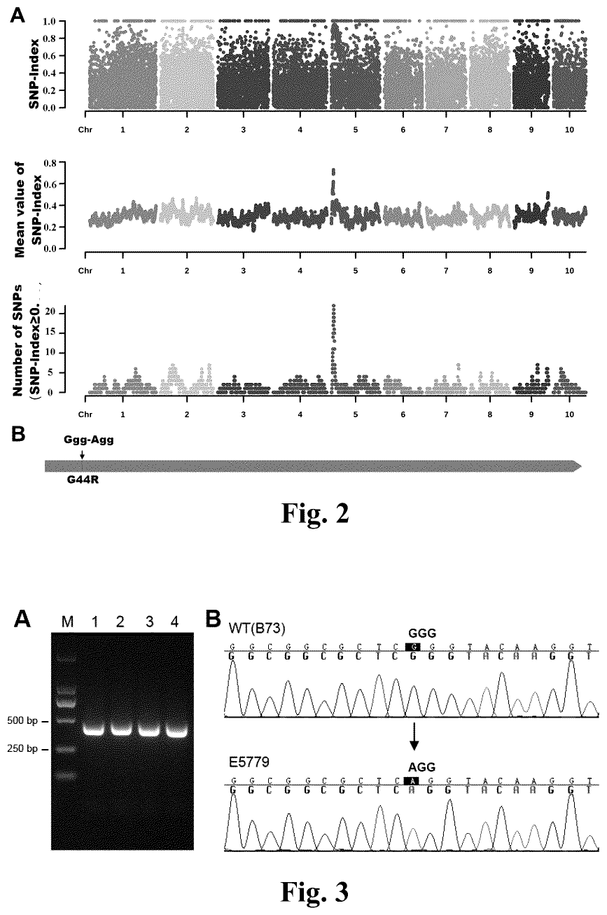

From the self-crossed F2 separation population of B73×E5779, 19 mutant phenotype individual plants are selected, a Tiangen plant genome DNA extraction kit (DP305-03) is used to extract DNA respectively, and the specific operation process is shown in kit instructions. DNA integrity and concentration are measured by agarose gel electrophoresis and Qubit (Invitrogen, USA), and qualified DNA is used for subsequent analysis. DNA of each individual plant is mixed equally, 1 μg of the mixed DNA is taken for creating a library by using an Illumina TruSeq DNA library creating kit and sequenced by using an Illumina NovaSeq 6000 sequencer. The quality control is performed for the raw reads obtained from sequencing, to obtain clean reads. The published B73 genome (v4.32) is used as a reference genome, and a Mutmap mapping method is used to map a mutated gene (Lu et. al., Mol Plant, 2018, 11, 496-504). The identified single nucleotide polymorphism (SNP) is screened according to G->A (C->T) base mutation mode and SNP-Index=1.0 standard. On this basis, the variation type of the screened SNP is analyzed, and stop gained/lost, splice-site acceptor/donor, start gained/lost, and non-synonymous coding are screened. By calculating and analyzing the mean value of SNP-Index within the 3 Mb upstream and downstream range of each SNP, a significant SNP signal site interval is identified, and results are shown in FIG. 2. Furthermore, combined with the analysis to PCR amplification, Sanger sequencing verification, and functional annotation results and the like of the individual plant in the F2 separation population, it is determined that the E5779 mutant is caused by a non-synonymous mutation from G to A occurring in the gene Zm00001d013465 within this interval, Zm00001d013465 encodes the maize Dwarf9 gene, the mutation causes the 44th amino acid of Dwarf9 to change from glycine (G) to arginine (R), and the mutation is located within a DELLA domain.

4. Molecular Marker Design for Wild-Type and E5779 Mutant

The genomic DNA of wild-type B73 inbred line and E5779 mutant is extracted respectively by using an Aikorei plant genome kit, and used for subsequent PCR analysis after it is qualified by electrophoresis detection. According to the site of the E5779 mutant gene, a specific primer is designed to amplify a sequence length of 439 bp, as shown in SEQ ID NO.3. The primer sequence is as follows:

SEQ ID NO. 4: E5779-F:
5′-GAGCTCGGACAGCATGCTCT-3′
SEQ ID NO. 5: E5779-R:
5′-TCCGTTTCCTTCCCAACTCCCAAT-3′.

A PCR amplification technology is used, and the specific primer is used to detect wild-type B73 and E5779 respectively. A PCR amplification system includes 12.5 μL of Taq Master Mix (2×), 1.0 μL of upstream and downstream primers, 2.0 μL of genomic DNA, and 8.5 μL of ddH₂O, and the total system is 25 μL. An amplification reaction procedure of PCR is as follows: it is pre-denaturated at 95° C. for 5 min, denaturated at 95° C. for 30 s, annealed at 65° C. for 30 s, extended at 72° C. for 40 s, totally extended at 72° C. for 10 min, and stored at 10° C. for 2 h. After the PCR reaction, 1.2% of agarose gel electrophoresis is used for detection. The size of a product is consistent with the size of a theoretical length, and the product is high in specificity and does not have a non-specific band. The PCR product is sent to a biological sequencing company for reverse primer sequencing, sequencing results are analyzed for the mutation site by using Sequencer software, the results show that the mutation site of the E5779 mutant is the base A, and the wild-type (B73) is the base G at the corresponding site (FIG. 3), which indicates that this marker may identify the wild-type and mutant genotypes and may be used for molecular breeding assisted molecular identification.

5. Breeding Application of E5779 Mutant

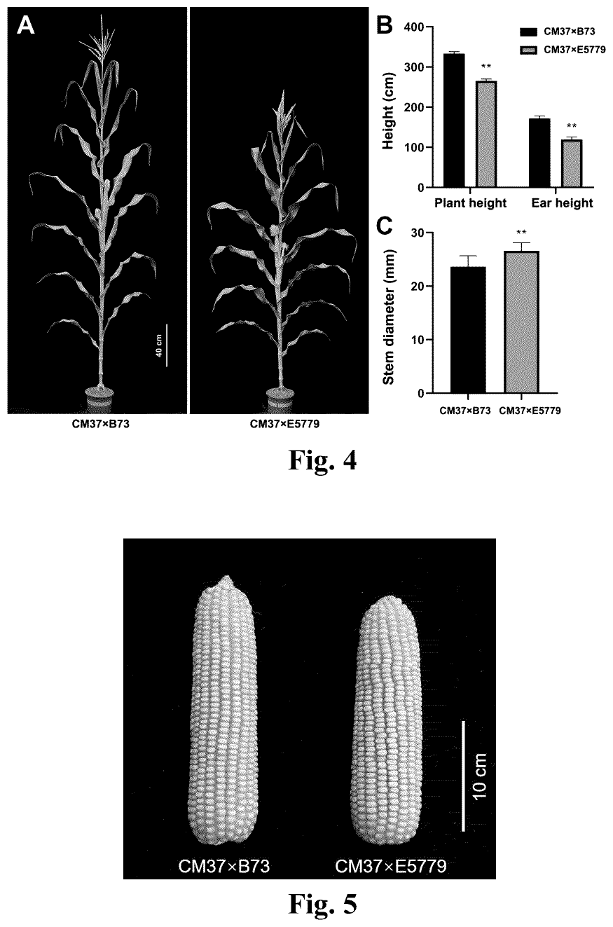

In order to evaluate the application of the E5779 mutant in breeding, the wild-type B73 and E5779 mutants are used as male parents, and hybridized respectively with a female parent CM37 (Xingen Zhang et. al., Comparative Transcriptomics Reveals the Molecular Mechanism of the Parental Lines of Maize Hybrid An'nong876 in Response to Salt Stress, Int J Mol Sci, 2022, 23, 5231.) of a new maize variety An'nong 876 (variety certification number: Guoshenyu 2020305) bred by the applicant, and then its phenotype is statistically analyzed. Results are shown in FIGS. 4 and 5, and FIG. 4 shows that compared to the CM37×B73 hybridized combination, the plant height (average decrease of 20.2%) and ear height (average decrease of 30.5%) of the CM37×E5779 hybridized combination are significantly reduced, and the improvement of the plant height and ear height traits is relatively ideal. It further indicates that the mutant may reduce the plant height of the F₁-generation and have the semi-dominant characteristics. At the same time, the stem of the CM37×E5779 hybridized combination is thickened. FIG. 5 shows that compared to the CM37×B73 hybridized combination, the CM37×E5779 hybridized combination shows better performance in ear capping, grain plumpness, ear length and other aspects. It indicates that the dwarf molecular marker has a relatively small effect on the yield of the F₁ plant (CM37×E5779), and it is further proved that E5779 is a mutant with potential for maize density-tolerant breeding and has an industrial promotion prospect.

The above are detailed implementation modes and specific operation processes of the present application, and are implemented under the premise of technical schemes of the present application. However, the scope of protection of the present application is not limited to the above embodiments.

1. A semi-dominant molecular marker related to maize dwarf, wherein the molecular marker is caused by a non-synonymous mutation from G to A in a maize gene as shown in SEQ ID NO: 1, the molecular marker has a nucleotide sequence as shown in SEQ ID NO: 3, and the non-synonymous mutation site is located in the 130th site of SEQ ID NO: 1.

2. A specific primer for detecting the molecular marker related to the maize dwarf as claimed in claim 1, wherein the sequence of the specific primers are:
E5779-F:
(SEQ ID NO: 4)
5′-GAGCTCGGACAGCATGCTCT-3′;
and
E5779-R:
(SEQ ID NO: 5)
5′-TCCGTTTCCTTCCCAACTCCCAAT-3′.

3. A method for identifying a molecular marker related to maize dwarf with the primers as claimed in claim 2, comprising the following steps: a PCR amplification of a genomic DNA of a maize plant to be identified with the primers to obtain an amplification product, sequencing the amplification product, and determining the base type of the non-synonymous mutation site of the molecular marker.

4. A method for breeding a new variety of dwarf and density-tolerant maize comprising an amplification of the molecular marker related to the maize dwarf as claimed in claim 1.

5. The method as claimed in claim 4, wherein the molecular marker has semi-dominance in a maize plant, the molecular marker is introduced into a plant of a variety to be improved by a field breeding or molecular breeding technology, a heterozygous dwarf plant with a genotype AG or a homozygous dwarf plant with a genotype AA is obtained, and the plant height satisfies: homozygous dwarf plant<heterozygous dwarf plant<maize plant to be improved.

6. The method as claimed in claim 5, wherein the method using the field breeding technology comprises the following steps: hybridizing a parent of the variety to be improved with a variety containing the molecular marker related to the maize dwarf, to obtain an F₁-generation heterozygous dwarf plant, self-crossing or back-crossing the F₁-generation heterozygous dwarf plant, and acquiring the homozygous dwarf plant or the heterozygous dwarf plant by trait separating and screening.

7. The method as claimed in claim 6, wherein the variety containing the molecular marker related to the maize dwarf is a maize E5779 dwarf mutant.

8. The method as claimed in claim 5, wherein the method using the molecular breeding technology comprises the following step: using a gene editing technology to mutate a base of the non-synonymous mutation site of the molecular marker in the genome of the variety to be improved from G into A, to obtain a dwarf variety.


SEQUENCE LISTING (5 sequences)

SEQ ID NO: 1 - DNA, 1878 bp, Zea mays
  source 1..1878: mol_type = other DNA
atgaagcgcgagtaccaaaacgccggcgggaacgacggctacaggggctcctccaaggacaagtcgatggcggcggcggcgggggcaggggagcaggaggaggaggtggacgagctgctggcggcgctcgggtacaaggtgcgttcgtcggatatggcggacgtcgcgcagaagctagagcagctcgagatggccatggggatgggcggcgcctgccccaccgctgatgacgggttcgtctcgcacctcgccacggacaccgtgcactacaatccctccgacctgtcgtcctgggtcgagagcatgctgtccgagctcaacgcgcccccgccgccgctcccacccgcgacgccggcaccaaggctggcgtccacctcgtccaccgtcacaagtggcgccgccgccggtgccggctacttcgatctcccgcccgccgtcgactcgtccagcagtacctacgctctgaagccgatcccctcgccggtggcggcggcgtcggccgacccgtccccggactcggcgcgggagcccaagcggatgcgaactggcggcggcagcacgtcgtcgtcctcttcctcgtcgtcatccatggacggcggccgcactaggagctccgtggtcgaagctgccccgccggcgacgcaggcggccaacgggccggcggtgccggtggtggtggtggacacgcaggaggccgggatccggctggtgcacgcgctgctggcgtgcgcggaggccgtgcagcaggagaacttctctgcggcggacgcgctggtgaagcagatccccgtgctggcctcgtcgcagggcggcgccatgcgcaaggtcgccgcctacttcggcgaggcgctcgcccggcgcgtgtatcgcctccgcccggcaccggacggctccctcctcgacgccgccttcgccgacctcctgcacgcgcacttctacgagtcctgcccctacctcaagttcgcccacttcaccgcgaaccaggccatcctcgaggctttcgccgggtgccgccgcgtccacgtcgtcgacttcggcatcaagcaggggatgcagtggccggctctcctccaggccctcgccctccgccccggcggccccccgtcgttccgtctcaccggcgtaggcccgccgcagcccgacgagaccgacgccctgcagcaggtgggctggaagctcgcccagttcgcgcacaccatccgcgtcgacttccagtaccgtggcctcgtcgccgccacgctcgctgacctggagccgttcatgctgcgaccggagggcggcggcgacacggacgacgagcccgaggtgatcgccgtaaactcggtgtgcgagctgcaccggctgctcgcgcagcccggtacactcgacaaggtcctgggcaccgtgcgcgcggtgcggccgaggatcgtgacggtggtggagcaggaggccaaccacaactccggcacattcctcgaccgcttcacggagtcgctgcactactactccaccatgttcgactccctcgagggcgccggctcaggctccggctccggctccggctccggccagcccaccgacgcctccccgccggccggcacggaccaggtgatgtccgaggtgtacctcggccggcagatctgcaacatcgtggcgtgcgagggcgccgagcgcacggagcgccacgagacgctggtccagtggcgcggccgcctcggcgggtccgggttcgagcccgtgcacctgggatccaacgcctacaagcaggcaagcacgctgctggccctcttcgccggcggcgacgggtacagggtggaggagaaggacgggtgcctgactctgggatggcatacgcgcccgctcatcgccacctcggcgtggcgcgtcgccgctccgtga

SEQ ID NO: 2 - DNA, 1878 bp, Zea mays
  source 1..1878: mol_type = other DNA
atgaagcgcgagtaccaaaacgccggcgggaacgacggctacaggggctcctccaaggacaagtcgatggcggcggcggcgggggcaggggagcaggaggaggaggtggacgagctgctggcggcgctcaggtacaaggtgcgttcgtcggatatggcggacgtcgcgcagaagctagagcagctcgagatggccatggggatgggcggcgcctgccccaccgctgatgacgggttcgtctcgcacctcgccacggacaccgtgcactacaatccctccgacctgtcgtcctgggtcgagagcatgctgtccgagctcaacgcgcccccgccgccgctcccacccgcgacgccggcaccaaggctggcgtccacctcgtccaccgtcacaagtggcgccgccgccggtgccggctacttcgatctcccgcccgccgtcgactcgtccagcagtacctacgctctgaagccgatcccctcgccggtggcggcggcgtcggccgacccgtccccggactcggcgcgggagcccaagcggatgcgaactggcggcggcagcacgtcgtcgtcctcttcctcgtcgtcatccatggacggcggccgcactaggagctccgtggtcgaagctgccccgccggcgacgcaggcggccaacgggccggcggtgccggtggtggtggtggacacgcaggaggccgggatccggctggtgcacgcgctgctggcgtgcgcggaggccgtgcagcaggagaacttctctgcggcggacgcgctggtgaagcagatccccgtgctggcctcgtcgcagggcggcgccatgcgcaaggtcgccgcctacttcggcgaggcgctcgcccggcgcgtgtatcgcctccgcccggcaccggacggctccctcctcgacgccgccttcgccgacctcctgcacgcgcacttctacgagtcctgcccctacctcaagttcgcccacttcaccgcgaaccaggccatcctcgaggctttcgccgggtgccgccgcgtccacgtcgtcgacttcggcatcaagcaggggatgcagtggccggctctcctccaggccctcgccctccgccccggcggccccccgtcgttccgtctcaccggcgtaggcccgccgcagcccgacgagaccgacgccctgcagcaggtgggctggaagctcgcccagttcgcgcacaccatccgcgtcgacttccagtaccgtggcctcgtcgccgccacgctcgctgacctggagccgttcatgctgcgaccggagggcggcggcgacacggacgacgagcccgaggtgatcgccgtaaactcggtgtgcgagctgcaccggctgctcgcgcagcccggtacactcgacaaggtcctgggcaccgtgcgcgcggtgcggccgaggatcgtgacggtggtggagcaggaggccaaccacaactccggcacattcctcgaccgcttcacggagtcgctgcactactactccaccatgttcgactccctcgagggcgccggctcaggctccggctccggctccggctccggccagcccaccgacgcctccccgccggccggcacggaccaggtgatgtccgaggtgtacctcggccggcagatctgcaacatcgtggcgtgcgagggcgccgagcgcacggagcgccacgagacgctggtccagtggcgcggccgcctcggcgggtccgggttcgagcccgtgcacctgggatccaacgcctacaagcaggcaagcacgctgctggccctcttcgccggcggcgacgggtacagggtggaggagaaggacgggtgcctgactctgggatggcatacgcgcccgctcatcgccacctcggcgtggcgcgtcgccgctccgtga

SEQ ID NO: 3 - DNA, 439 bp, Zea mays
  source 1..439: mol_type = other DNA
gagctcggacagcatgctctcgacccaggacgacaggtcggagggattgtagtgcacggtgtccgtggcgaggtgcgagacgaacccgtcatcagcggtggggcaggcgccgcccatccccatggccatctcgagctgctctagcttctgcgcgacgtccgccatatccgacgaacgcaccttgtacccgagcgccgccagcagctcgtccacctcctcctcctgctcccctgcccccgccgccgccgccatcgacttgtccttggaggagcccctgtagccgtcgttcccgccggcgttttggtactcgcgcttcatgatctcggagctacaggccgggctagccagctaataattgcttgcgcccctccgtatcggttccgggatagcttgggatttggatccagggttgggaattgggagttgggaaggaaacgga

SEQ ID NO: 4 - DNA, 20 bp, synthetic construct
  source 1..20: mol_type = other DNA
gagctcggacagcatgctct

SEQ ID NO: 5 - DNA, 24 bp, synthetic construct
  source 1..24: mol_type = other DNA
tccgtttccttcccaactcccaat